(19) United States
(12) Patent Application Publication
(10) Pub. No.: US 2026/0306081 A1
(43) Pub. Date: Oct. 8, 2026

(54) A DEVICE FOR EXTRACTING A MATERIAL

(71) Applicant: Stichting Radboud universitair medisch centrum, Nijmegen (NL)

(72) Inventor: Dennis George Hugo Bosboom, Kranenburg/Nutterden (DE)

(73) Assignee: Stichting Radboud universitair medisch centrum, Nijmegen (NL)

(21) Appl. No.: 19/482,900

(22) PCT Filed: May 8, 2024
(86) PCT No.: PCT/EP2024/062846

§ 371 Date: Nov. 10, 2025

(30) Foreign Application Priority Data
May 11, 2023 (EP) 23172942.7

(51) Int. Cl. A61B 10/02; A61B 17/32

(52) U.S. Cl. CPC A61B 10/0266; A61B 17/32002; A61B 2010/0208; A61B 2017/320064

(57) ABSTRACT

A device and a method for material extraction during medical procedures such as biopsy, drainage, surgery, ablation, or any procedure that involves extracting unwanted material from an extraction site at the human or animal body. The device may be also used to deliver material to a delivery site. The device comprising a tubular member having a lumen and a series of radially inward projections extending into the lumen, and a shaft movably arranged within the lumen of the tubular member and having a series of radially outward projections extending into the lumen.

### FIELD OF THE INVENTION

The invention relates to a device for extracting a material. The invention further relates to a method for material transport.

### BACKGROUND OF THE INVENTION

Extraction of biological material from the body for laboratory testing is known as biopsy. Biopsies are often performed for the diagnosis and treatment of patients with malignant lesions. Extracting internal biological material, or tissue, involves the use of devices typically consisting of a tubular member, such as a needle, configured to extract a pre-determined amount of material, namely a tissue sample. To better characterize the biological material, multiple samples may be extracted. With typical devices, the first tissue sample may be collected outside the body and the needle may be re-inserted. A second insertion may cause further harm to the tissue surrounding the extraction site and increase the cost of the procedure, as a new needle may be used for the second sample.

U.S. Pat. No. 8,951,207B2 describes a device comprising a needle and a cutter to remove tissue from an extraction site. A vacuum source operates with the cutter to pull the tissue into a tissue collection chamber. However, this solution involves a complex mechanism associated to a high cost of the device. In general, there is the need to for an improved device for material extraction.

### SUMMARY OF INVENTION

It would be advantageous to provide an improved device for extracting a material.

Therefore, according to a first aspect of the invention, there is provided a device for extracting a material, comprising:
a tubular member having a lumen and a series of inward projections extending into the lumen, and
a shaft movably arranged within the lumen of the tubular member, and having a series of outward projections extending into the lumen,
wherein either one of the outward projections and the inward projections are first projections, the first projections having a helical shape with a pitch and a space in axial direction between successive windings of the helical shape, and the other one of the outward projections and the inward projections are second projections,
wherein the second projections are configured to extend at least partially into the space between the successive windings, and wherein first slots between adjacent ones of the first projections allow the second projections to pass through first slots of the first projections, and second slots between adjacent ones of the second projections allow the first projections to pass through the second slots of the second projections.

The device for extracting a material can be made of a small number of components that allows a simple and cost-efficient extraction of a desired amount of material without displacing the tubular member from an extraction site located, for example, in deep tissues like internal organs. Relative rotation of the shaft and the tubular member around their longitudinal axis allows the first projections to remove material from an extraction site by virtue of their helical shape. Simultaneously, the second projections, by virtue of their extension at least partially into the space between the first projections, scrap the removed material from the first projections to retain it in the tubular member. During rotation, the first and second projections pass each other such that a new amount of material is removed from the extraction site and the extracted material is transported in axial direction within the tubular member. The first projections may pass in a helical direction through the second slots situated between adjacent ones of the second projections, which allow a smooth rotation of the shaft within the lumen.

The second projections may be arranged in at least one axially oriented row, wherein the second projections of the row are attached at a same circumferential position on an inner surface of the tubular member or an outer surface of the shaft. This provides an easy-to-use configuration, where rotational movement may be alternated with axial movement of the shaft with respect to the tubular member. Two examples of this configuration are as follows.

The series of inward projections may be arranged in at least one axially oriented row, wherein the inward projections of the row of inward projections are attached to an inner surface of the tubular member at a same circumferential position on the inner surface of the tubular member. This advantageously may enable transport of the removed material within the tubular member axially in a proximal direction.

The series of outward projections may be arranged in at least one axially oriented row, wherein the outward projections of the row of outward projections are attached to an outer surface of the shaft at a same circumferential position on the outer surface of the shaft. This ensures a consistent way to remove material by the outward projections along the shaft.

The first slots between the first projections may be arranged in at least one first axially oriented row and the second slots between the second projections may be arranged in at least one second axially oriented row. Advantageously, the second slots provide guidance to the helical first projections when the shaft is being rotated, therewith inducing an axial movement of the shaft at every turn, resembling the function of an internal thread. In this manner, the second slots facilitate the removal of material as the shaft, or the tubular member, rotates and advances simultaneously.

The inward and/or outward projections may be blades. Advantageously, blades provide improved cutting properties, which prevents further damage to the surrounding tissues at the extraction site, thus facilitating a more effective material extraction. Blades may be suitable for guiding the material through the tubular member.

The second projections may have a rectangular shape. Advantageously, a rectangular shape is simpler to manufacture. They may be oriented in an angle alpha from a longitudinal axis of the tubular member. The orientation of the second projections may be adapted depending on the angle of the windings of the first projections, such to improve the scraping of material.

In some embodiments, each winding of the helical shape of the first projections is formed by a fixed number of first projections. In this context, a winding corresponds to a complete turn of a helix (360 degrees). A winding may be formed by one first projection having a helical shape extending 360 degrees or may be formed by two or more first projections next to each other. For example, a winding may comprise two or three projections (which may be equally sized), with slots therebetween.

The tubular member may comprise inner distal and proximal annular stoppers to stop, respectively, extension and retraction of an outer ring mounted on the circumferential surface of a proximal portion of the shaft. Advantageously, the annular stoppers and the outer ring limit the amount of axial displacement of the shaft relative to the tubular member, which ensures a correct assembly of the device during use and ensures that the extracted material is contained in the tubular member. For example, the stoppers may be provided in a proximal portion of the tubular member, proximal with respect to the projections.

In some embodiments, the device may comprise an elastic element between one of the annular stoppers of the tubular member and the outer ring of the shaft. An elastic element may provide a bias between the tubular member and the shaft. An elastic element may provide the advantage to assist either the extension or retraction of the shaft relative to the tubular member, facilitating the use of the device.

The second projections may also have a helical shape and may have a rotational direction opposite to the rotational direction of the helix of the first projections, which simplifies handling of the shaft and tubular member to extract material. Rotating the shaft or the tubular member in a first direction may remove material from the extraction site and transport the removed material proximally within the tubular member. In this manner, a desired amount of material can be extracted by only rotating the shaft or tubular member relative to each other. Abovementioned embodiment may further comprise an actuated mechanism to rotate the shaft and the tubular member around their longitudinal axis, which offers the advantage of possible robotization of and/or more reproducible or accelerated extraction of material.

The second projections may have a projected axial length corresponding substantially to the space in axial direction between the successive first projections. Advantageously, filling the axial space between successive first projections provides a more effective scrapping of material from the first projections.

The outward projections may extend radially outward to substantially cover a radius of the lumen of the tubular member from the outside surface of the shaft to the inner surface of the tubular member. This minimizes sticking of removed material to the inner wall of the tubular member, improving efficiency of material extraction.

The inward projections may extend radially inward to substantially cover the radius of the lumen of the tubular member from the inner surface of the tubular member to the outside surface of the shaft. Covering the radius of the lumen with the inward projections further improves scrapping of the removed material, minimizing sticking to the surface of the shaft.

The proximal portion of the tubular member may have a side aperture. Advantageously, the aperture provides an exit to the extracted material for collection outside the device, this allows to extract a large amount of material without withdrawing the device from the extraction site. In some embodiments, the inward projections may be disposed from a distal end of the tubular member to at least an axial location of the aperture, which ensures that the removed material is transported over the length of the tubular member from the extraction site to the aperture.

In some embodiments, the tubular member and the shaft may be configured such that material may be transported in both proximal and distal directions, thereby reversing the movements of the tubular member with respect to the shaft. As a result, the device may be used for procedures in which material is inserted or introduced in the human or animal body, such as brachytherapy. The space between the successive windings may be prefilled/or fed with radioactive seeds to be introduced into the body. Advantageously, the device for material insertion allows for a faster and continuous introduction of seeds with one insertion of the tubular member.

According to another aspect, a method is provided for material transport. The method comprises:
a step of providing a tubular member and a shaft in a lumen of the tubular member, the tubular member having a series of inward projections extending into the lumen, the shaft having a series of outward projections extending into the lumen, wherein either one of the outward projections and the inward projections are first projections, the first projections having a helical shape with a pitch and a space (SP) in axial direction between successive windings of the helical shape, and the other one of the outward projections and the inward projections are second projections, wherein the second projections extend at least partially into the space (SP) between the successive windings,
a first moving step of moving the shaft with respect to the tubular member so that the first projections pass through second slots between adjacent ones of the second projections, and
a second moving step of moving the shaft with respect to the tubular member so that the second projections pass through first slots between adjacent ones of the first projections.

The first moving step may remove material from the extraction site. If there is material already in the lumen of the tubular member, the first moving step will cause the second projections to scrap material from the first projections or the inner surface of the tubular member, preventing binding of material and allowing its transport within the tubular member. The second moving step may transfer the removed material into the lumen of the tubular member. If there is material already retained in the lumen of the tubular member, the second moving step will cause the retained material to be transported proximally by the first projections. The first and second step may be performed repeatedly, wherein the first step is followed by the second step and the second step is followed again by the first step. The method may extract material from an extraction site in the body, for example to take a biopsy, by providing the distal end of the tubular member at the extraction site and performing the movements to transport the material from the distal end in proximal direction through the tubular member. Alternatively, the method may deliver material to a delivery site in the body, for example to deliver medication to the delivery site, by providing the distal end of the tubular member at the delivery site and performing the movements to transport the material towards the distal end.

The amount of movement of the shaft with respect to the tubular member, at the first and the second moving step, may vary depending on the embodiment, the depth of the extraction/delivery site and the desired amount of material to be extracted/delivered considering the diameter of the tubular member and the size of the first and second projections.

The first moving step may comprise rotating the shaft until the first slots align with the second slots, and then proceeding with the second moving step. Rotating the shaft relative to the tubular member may remove material from the extraction site by virtue of the helical shape of the first projections. Advantageously, rotating the shaft until the first slots align with the second slots facilitates to repeat the first moving step or to perform the second moving step.

The second moving step may comprise moving the shaft until the first slots align with the second slots, and then proceeding with the first step. Advantageously, moving the shaft until the first slots align with the second slots facilitates to repeat the second moving step or to perform the first moving step. In embodiments in which the second projections have a rectangular shape and are oriented parallel (i.e., an angle alpha of 0 degrees) from a longitudinal axis of the tubular member, the second moving step may involve sliding the shaft axially within the lumen of the tubular member. In embodiments in which the second projections also have a helical shape with a rotational direction opposite to the rotational direction of the helix of the first projections, the second moving step may involve rotating the shaft around its longitudinal axis within the tubular member. This simplifies handling of the shaft and tubular member to extract material, as only rotation of the shaft and/or tubular member is needed to remove material.

The person skilled in the art will understand that the features described above may be combined in any way deemed useful. Moreover, modifications and variations described in respect of the device for material extraction may likewise be applied to the method, and modifications and variations described in respect of the method may likewise be applied to the device for material extraction.

### BRIEF DESCRIPTION OF DRAWINGS

In the following, aspects of the invention will be elucidated by means of examples, with reference to the drawings. The drawings are diagrammatic and may not be drawn to scale. Throughout the drawings, comparable items may be marked with the same reference numerals.

FIG. 1A shows a perspective view of a distal portion of a tubular member from a first example of a device for extracting a material.

FIG. 1B shows a side view of a distal portion of a shaft from a first example of a device for extracting a material.

FIG. 2 shows a longitudinal cross section of the tubular member of FIG. 1.

FIG. 3A-G illustrate steps of methods of extracting a material by using the first example of a device for extracting a material.

FIG. 4A shows a perspective view of a distal portion of a shaft from a second example of a device for extracting a material.

FIG. 4B shows an perspective view of a distal portion of a tubular member from a second example of a device for extracting a material.

FIG. 4C shows the shaft of FIG. 4A in the lumen of the tubular member of FIG. 4B.

FIG. 5 shows longitudinal cross sections of the tubular member of FIG. 4B.

The figures are meant for illustrative purposes only, and do not serve as restriction of the scope or the protection as laid down by the claims.

### DETAILED DESCRIPTION OF EMBODIMENTS

Certain exemplary embodiments will be described in greater detail, with reference to the accompanying drawings. The matters disclosed in the description, such as detailed construction and elements, are provided to assist in a comprehensive understanding of the exemplary embodiments. Accordingly, it is apparent that the exemplary embodiments can be carried out without those specifically defined matters. Also, well-known operations or structures are not described in detail since they would obscure the description with unnecessary detail. Certain embodiments of the present disclosure can be used during medical procedures such as biopsy, drainage, surgery, and ablation, etc. Certain embodiments of the present invention relate to a biopsy needle or a needle for minimally invasive treatment of a human or animal. Yet, the present invention is suitable for any procedure that involves extracting unwanted or diseased biological or even foreign material from an extraction site at the human or animal body.

The examples and embodiments described herein serve to illustrate rather than limit the invention. The person skilled in the art will be able to design alternative embodiments without departing from the scope of the present disclosure, as defined by the appended claims and their equivalents. Reference signs placed in parentheses in the claims shall not be interpreted to limit the scope of the claims. Items described as separate entities in the claims, or the description may be implemented as a single hardware item combining the features of the items described.

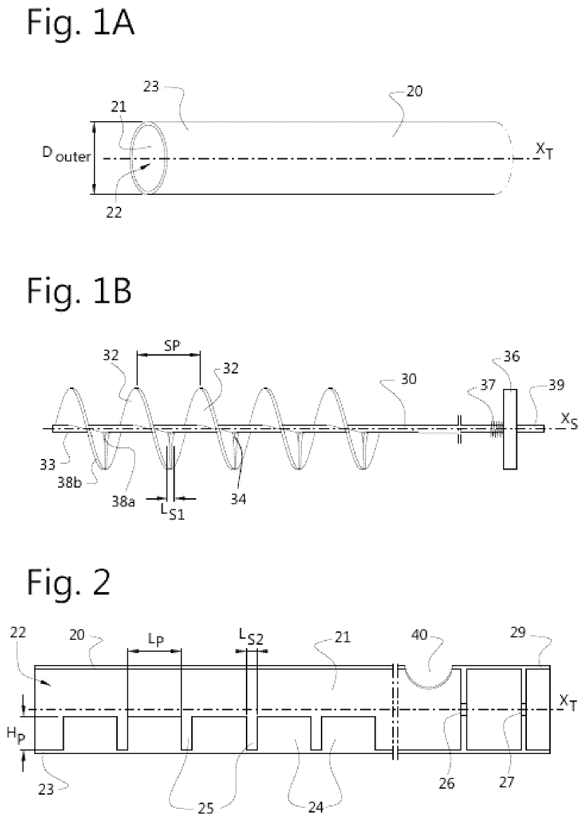

FIGS. 1A and 1B show a first example of a device 10, for example a biopsy device, for extracting a material according to the present disclosure. The device 10 comprises a tubular member 20 (FIG. 1A) having a cylindrical shape and an inner wall surface 21 enclosing a lumen 22. The device 10 further comprises a shaft 30 (FIG. 1B) configured to move within the lumen 22 of the tubular member 20 and having a series of outward projections 32, here also called first projections. The inner and outer diameter of the tubular member 20 may be adapted depending on the application. For minimally invasive medical procedures, e.g., biopsy, the outer diameter Dₒᵤₜₑᵣ may be between 3 mm and 5 mm with a wall thickness of about 0.3 mm. For other procedures that may involve a larger amount material to be extracted, e.g., diseased tissue removal, the outer diameter of the tubular member 20 may be between 5 mm and 15 mm. The length of the tubular member 20 may be between about 90 mm and 150 mm. The person skilled in the art will understand that the dimensions of the shaft 30 can be adapted according to the dimensions of the tubular member 20. The tubular member 20 may be made from a sheet of medical grade stainless steel or other material known in the art. The manufacture of the tubular member 20 may involve any process known in the art, for example, rolling the sheet and then welding into a fine tube. Consecutively, the fine tube may be stretched to the specified diameter and wall thickness. The shaft 30 may also be made from medical grade stainless steel and manufactured by any process known in the art, for example, additive manufacturing or die casting. For die casting, the steel may be first heated until it is molten and then drawn through one or more dice that will form the outward projections 32.

In certain embodiments, the tubular member 20 and the shaft 30 may be substantially stiff or slightly flexible or may have different degrees of flexibility over their length, for example, being a distal portion less flexible than a proximal portion to effectively penetrate superficial and deep tissues. Moreover, the tubular member 20 may have substantially the shape of a straight tube, e.g., to easily direct a distal end 23 towards the extraction site.

FIG. 1B shows a side view of the shaft 30. The outward projections 32 are arranged after each other on the outer surface of the shaft 30 forming an axially oriented row. In this example, the outward projections 32 have a helical shape and are placed at the same circumferential position of the outer surface of the shaft 30 such to form a helix with a plurality of windings along the shaft 30. The helix has a pitch that defines a space SP in axial direction between successive windings of the helix formed by the outward projections 32. Adjacent outward projections 32 are separated by slots 34 of length L_{S1}, here also called first slots. The slots 34 may also have a short helical shape.

As the outward projections 32 are identical to each other and equidistantly separated, the slots 34 are arranged in an axially oriented row at a same circumferential position of the outer surface of the shaft 30. In other embodiments, the outward projections 32 may be placed at a different circumferential position of the outer surface of the shaft 30, which may change the size of the slots (L_{S1}) or intermittently reduce the space SP between windings.

The outward projections 32 extend radially outward from a longitudinal axis X_{S} to substantially cover a radius of the lumen 22 of the tubular member 20 from the outside surface of the shaft 30 to the inner surface 21 of the tubular member 20. In other embodiments, the outward projections 32 may extend radially outward to a lesser extent from the outside surface of the shaft 30 to the inner surface 21 of the tubular member 20, depending on the composition and size of the extracted material. This can be possible when manufacturing process used to produce the device have relatively large tolerance. The radial 38a and/or circumferential edges 38b of the outward projections 32, at both sides of the slots 34, may be sharpened or have a thickness smaller that the thickness of the body of the outward projections 32 such to form blades, which facilitates the removal and extraction of material.

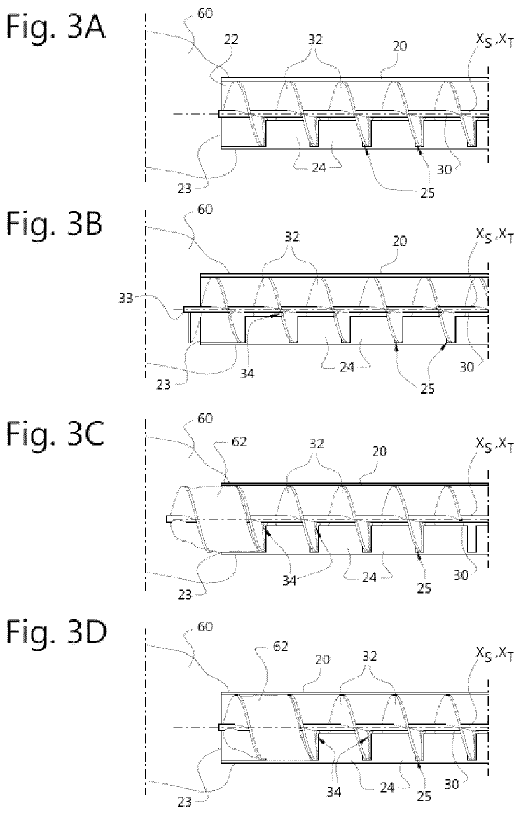

At a proximal portion 39, an annular ring 36 may be mounted on the circumferential surface of the shaft 30. The ring 36 may be configured to reside between an inner distal annular stopper 26 and a proximal annular stopper 27 of the tubular member 20 (See FIG. 2), thereby limiting the axial displacement of the shaft 30 relative to the tubular member 20. Optionally, an elastic element 37, e.g., a spring, between the annular ring 36 and one of the stoppers 26, 27 provides a bias between the tubular member 20 and the shaft 20. The direction of the bias depends on the position of the elastic element 37 relative the ring 36. For example, the elastic element 37 positioned distally from the ring 36 and proximally from the distal stopper 26 can assist retraction of the shaft 30 after it has been extended.

FIG. 2 shows a longitudinal cross section of the tubular member 20 of FIG. 1. The tubular member 20 comprises a series of inward projections 24, here also called second projections, extending into the lumen 22. The inward projections 24 are attached to an inner surface 21 of the tubular member 20. In this example, the inward projections 24 have a rectangular or prismatic shape with an axial length L_{P} and a height H_{P}. The height H_{P} defines the amount of inward extension into the lumen 22. In this example, H_{P} approaches the radius of the lumen 22, such to substantially cover the space between the inner surface 21 of the tubular member 20 and an outside surface of the shaft 30. In this manner, the inward projections 24 prevent material to bind to the outward projections 32, facilitating the extraction of semisolid and viscous material. It will be understood that the inward projections 24 may deviate from the rectangular or prismatic shape and still achieve the function of scrapping material from the outward projections 32 and retain it in the tubular member 20. In this example, the inward projections 24 are oriented in an angle alpha (α) of 0 degrees from a longitudinal axis X_{T} of the tubular member 20. A different orientation may be suitable depending on the angle of the windings of the outward projections 32, such to improve scraping of material. In other embodiments, the height H_{P} of the inward projections 24 may be smaller than the radial space between the shaft 30 to the inner surface 21 of the tubular member 20, which can be convenient if the composition and size of the extracted material is less likely to bind to the inner surface 21 of the tubular member 20 or when the used manufacturing process has a large tolerance.

The inward projections 24 are aligned at the same circumferential position on the inner surface 21 of the tubular member 20, such that they form an axially oriented row, parallel to the longitudinal axis X_{T} of the tubular member 20. Adjacent inward projections 24 are separated by slots 25 of length L_{S2}, also called second slots. The row of inward projections 24 is disposed from the distal end 23 of the tubular member 20 up until a side aperture 40 at a proximal portion 29 of the tubular member 20. The aperture 40 allows collection of the extracted material. Proximal to the aperture 40, the inner distal 26 and proximal 27 annular stoppers are disposed to stop, respectively, extension and retraction of the outer ring 36 mounted on the shaft 30. The aperture 40 may be located at the proximal end of the tubular member 20. In other embodiments, the proximal portion 29 of the tubular member 20 may comprise a container to collect the extracted material at a side or a proximal end. In some embodiments, the tubular member 20 may have a beveled or sharpened distal end 23 to facilitate penetration of the extraction site.

The person skilled in the art will understand that that the features described above can lead to other embodiments also covered by the present disclosure. For instance, the first example described with reference to FIG. 1 and FIG. 2 may be implemented with the shape of the inward and outward projections inverted, i.e., the inward projections having a helical shape and the outward projections having a prismatic shape. In this alternative example, the inward projections may be the first projections and the outward projections may be the second projections.

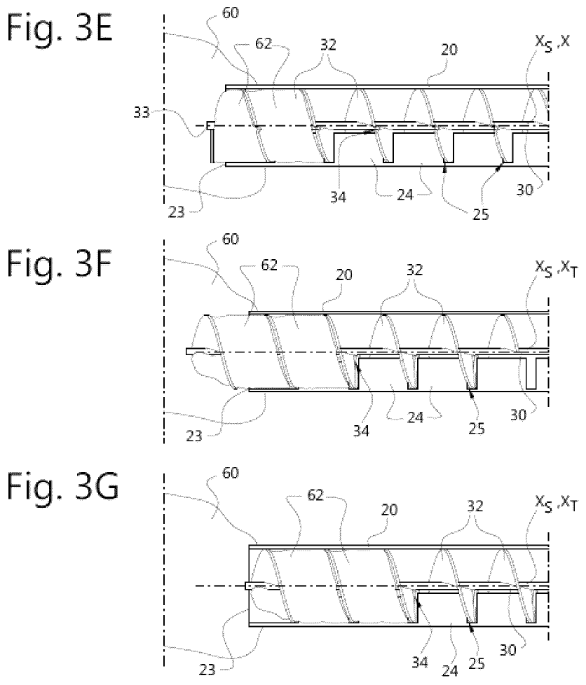

Another aspect of the present disclosure relates to a method to extract material from an extraction site, as exemplified by FIGS. 3A-G. In this context, the extraction site 60 may located, for example, at deep or superficial parts of the human or animal body. This method preferably used with the device of FIG. 1. The method may comprise:
providing a tubular member 20 and a shaft 30 in the lumen 22 of the tubular member 20 at an extraction site 60 (FIG. 3A). The tubular member 20 having a lumen 22 and a series of inward projections 24 extending into the lumen 22. The shaft 30 having a series of outward projections 32 extending into the lumen 22. The outward projections 32 having a helical shape with a pitch and a space SP in axial direction between successive windings of the helical shape, wherein the inward projections 24 are configured to extend at least partially into the space SP between the successive windings, and wherein slots 34 between adjacent ones of the outward projections 32 allow the inward projections 24 to pass through slots 34 of the outward projections and slots 25 between adjacent ones of the inward projections 24 allow the outward projections 32 to pass through the slots 25 of the second projections.
a first moving step of rotating the shaft 30 with respect to the tubular member 20, around its longitudinal axis X_{S} such that the tubular member 20 advances in distal direction, and its distal end 33 may protrude from the distal end 23 of the tubular member 20 (FIG. 3B). As the shaft 30 rotates, the outward projections 32 pass through the slots 25 between adjacent ones of the inward projections 24. The slots 25 provide guidance to the outward projections 32 when the shaft 30 is being rotated, therewith inducing an axial movement of the shaft 30 at every turn. In this manner, the slots 25 facilitate the removal of material as the shaft 30 rotates and slides simultaneously. As the shaft 30 is being rotated, the outward projections 32 penetrate and remove material 62 (or tissue sample) from the extracting site by virtue of their helical shape. In the example shown, each winding is formed by one of the outward projections 32 extending about 360 degrees. Thus, the shaft 30 is rotated about 360 degrees until the slots 34 between outward projections 32 and the slots 25 between inward projections align axially (FIG. 3C). The skilled person will understand that each winding of the helical shape can be formed by any number of outward projections 32. For instance, having windings formed by two or more outward projections 32 provides the advantage to reduce the amount of rotation needed to remove material before it is transferred in the tubular member 20. This can be convenient when the extraction site 60 has a small depth or when going deeper is troublesome. For example, when the extracting material is nearby tissues that should not be affected.
a second moving step of sliding the shaft 30 proximally to an amount corresponding to the projected axial length L_{P} of at least one of the inward projections 24 such that the outward projections 32 align with the space SP between the successive windings and the slots 34 between outward projections 32 align with the slots 25 between inward projections 24 (FIG. 3D). In this example, the projected axial length L_{P} is equal to the axial length of the outward projections 32. The slots 34 between outward projections 32 allow the shaft 30 to move in axial direction within the tubular member 20 without the inward projections 24 blocking the outward projections 32. The shaft 30 may be retracted to a desired amount. In this example, the shaft is retracted a distance corresponding to the projected axial length L_{P} of one of the inward projections 24, effectively transferring the removed material 62 into the tubular member 20 and preparing the shaft 30 to rotate again.

The first moving step of rotating the shaft 30 may be performed again to extract more material. During rotation (FIG. 3E), the inward projections 24 can scrap the removed material 62 from the outward projections 32, allowing it to remain in the tubular member 20, while the distal end 33 of the shaft 30 protrudes and removes a new amount of material 64 (FIG. 3F). As mentioned before, the shaft 30 may be rotated until the slots 34 between outward projections 32 and the slots 25 between inward projections align axially. At this position, the shaft 30 may rotate again or then may be retracted to transfer the new removed material 64 into the tubular member 20 and push in axial direction the retained material 62 (FIG. 3G). The steps of rotating and retracting the shaft 30 may be repeated until the desired amount of material is extracted.

To deliver material to the distal end, the movements may be reversed in direction. That is, in the first moving step, the shaft 30 may be rotated with respect to the tubular member 20, around its longitudinal axis X_{S} such that the tubular member 20 advances in proximal direction, and in the second moving step, the shaft 30 may slide in distal direction by an amount corresponding to the projected axial length L_{P} of at least one of the inward projections 24.

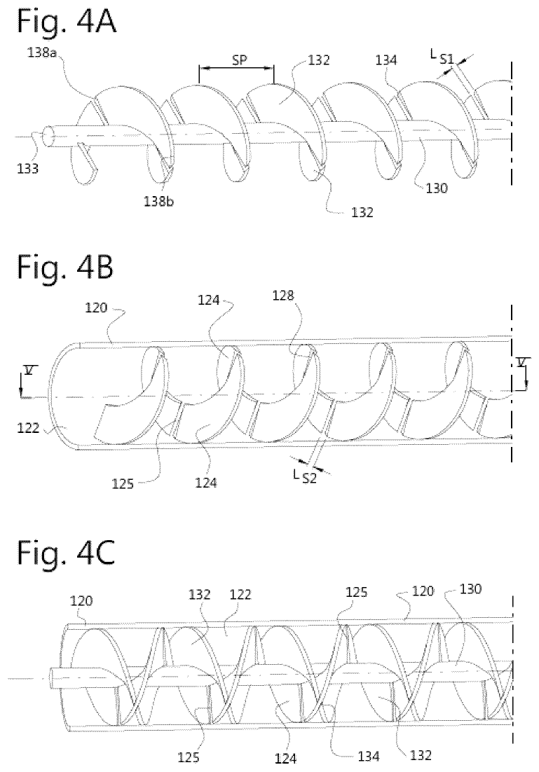

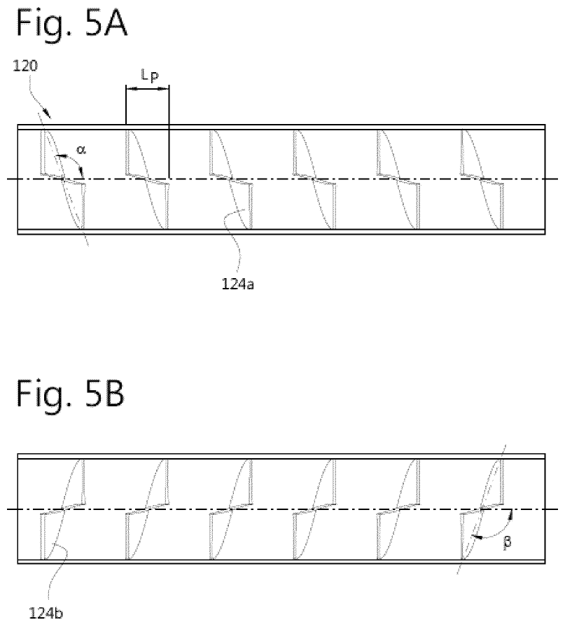

FIGS. 4A, 4B and 4C show a second example of a device for extracting a material according to the present disclosure. The device 110 comprises a tubular member 120 (FIG. 4B) having an inner wall 121 enclosing a lumen 122, and a shaft 130 (FIG. 4A). FIG. 4C shows a distal portion of the assembled device 110, wherein the shaft 130 of FIG. 4A is provided in the lumen 122 of the tubular member 120 of FIG. 4B. Features of the device that have already been described above with reference to the first example may also be present in the device shown in FIGS. 4 and 5 and will not all be described here again, features are designated with similar reference numerals preceded by 100 to distinguish the embodiments.

As shown in FIG. 4A, the shaft 130 comprises outward projections 132 arranged after each other on the outer surface of the shaft 130 forming an axially oriented row. In this example, each winding of the helical shape is formed by two outward projections 132, each extending about 180 degrees and having slots 134 of size L_{S1} therebetween, wherein helical slots 134 between outward projections 132 are arranged in axially oriented rows. The radial 138a edges of the outward projections 132 have a triangular end to facilitate removal of material and passage of the inward projections 124. The radial 138a or circumferential 138b edges of the outward projections 32, at both sides of the slots 134, may be sharpened or have a thickness smaller that the thickness of a body of the outward projections 132 such to form blades, which facilitates the removal of material.

FIG. 4B shows an open side view of a distal portion of the tubular member 120. In the depicted example, the inward projections 124 also have a helical shape forming a helix of the same pitch as the outward projections 13 but with an opposite rotational direction. Similar to the outward projections 132, each winding of the helical shape is formed by two outward projections 124, each extending about 180 degrees and having helical slots 125 of size L_{S2} therebetween. In this manner, inward projections 124 can pass the outward projections 132 when at least one of the tubular member 120 and the shaft 130 rotates along its longitudinal axis (X_{T}, X_{S}). The radial 128 edges of the inward projections 124 have a triangular end to facilitate removal of material and passage of the outward projections 132.

FIG. 5 shows longitudinal cross sections of the tubular member of FIG. 4B depicting a first half (FIG. 5A) and a second half (FIG. 5B) of the tubular member 120. The inward projections 124 have a projected axial length L_{P} corresponding substantially to the space SP in axial direction between the successive outward projections 124, which provides an effective scraping of material. The helix formed by the inward projections 124 gives to the first half projections 124a (FIG. 5A) an angle alpha (α) and to the second half projections 124b (FIG. 5B) an angle beta (β).

As explained before, another aspect of the present disclosure relates to methods for transporting material through a tubular member. For example, for extracting material from an extraction site or providing material to a delivery site. The following method is preferably used with the second example of the device described with reference to FIG. 4 and FIG. 5. As for the apparatus, features of the method that have already been described above with reference to the first example and FIG. 3 may also be present in the method below and will not all be described here again. The method may comprise:
providing a tubular member 120 and a shaft 130 in the lumen 122 of the tubular member 120. The tubular member 120 having a lumen 122 and a series of inward projections 124 extending into the lumen 122. The inward projections 124 having a helical shape with a pitch and a space in axial direction between successive windings of the helical shape. The shaft 130 having a series of outward projections 132 extending into the lumen 22. The outward projections 32 having a helical shape with the same pitch and a space as the inward projections 124 and with a rotational direction opposite to the rotational direction of the helix of the inward projections 124, wherein the inward projections 124 are configured to extend at least partially into the space between the outward projections 132, and wherein slots 134 between adjacent ones of the outward projections 132 allow the inward projections 124 to pass through slots 134 of the outward projections and slots 125 between adjacent ones of the inward projections 124 allow the outward projections 132 to pass through the slots 125 of the second projections.
a first moving step of rotating the shaft 130 or the tubular member 120, around its longitudinal axis (X_{S} or X_{T}). As the shaft 130 or the tubular member 120 rotate, the shaft 130 moves in a first axial direction with respect to the tubular member 120 and the outward projections 132 pass through the slots 125 between adjacent ones of the inward projections 124. The outward projections 132 may remove material (or tissue sample) from the extracting site. The shaft 130 may be rotated until the slots 134 between outward projections 132 and the slots 125 between inward projections 124 align such that the first moving step can be repeated, or another movement can be performed.
a second moving step of rotating the shaft 130 or the tubular member 120, around its longitudinal axis (X_{S} or X_{T}), wherein the rotation direction is the same in the first and second moving steps. As the shaft 130 or the tubular member 120 rotate in the second moving step, the shaft 130 moves in a second axial direction with respect to the tubular member 120, the second axial direction being opposite to the first axial direction. Moreover, the inward projections 124 pass through the slots 130 between adjacent ones of the outward projections 132. The shaft 130 may be rotated until the slots 134 between outward projections 132 and the slots 125 between inward projections align such that either the first or second moving step can be repeated. The slots 125, 132 between outward and inward projections 132, 124 allow the shaft 130 and the tubular member 120 to intermittently move in opposite axial directions, while keeping the rotation direction constant, which makes the current example suitable for an easy robotized implementation with a motorized mechanism at the proximal portion of the device.

A motorized mechanism may be arranged at the proximal portion of the device to move the tubular member 120 and/or the shaft 130 according to the first and second moving step repeatedly. In the second example, shown in FIG. 4 and FIG. 5, both the first and second moving step may involve rotation of the shaft with respect to the tubular member around their longitudinal axis, and the rotation may be in the same direction in both the first and second steps. Therefore, a simple actuator or motor (not illustrated) may provide such a rotary movement. The actuator may be provided, for example, at a proximal end of the biopsy device and may be controlled, for example, by a button. The mechanism may further comprise one or more elastic elements providing unidirectional or bidirectional bias in axial direction between the tubular member 120 and the shaft 130. The elastic elements may provide the advantage to guide the second projections into the first slots after the first moving step, to start the second moving step. Likewise, the elastic elements may guide the first projections into the second slots after the second rotating step, to start the first step. This way the extraction may be motorized. It will be understood that the bias may also be convenient in a manually rotated device.

Any of the above-mentioned examples of a device for material extraction can be operated manually or it can be actuated. Manual operation provides a more affordable device making it easily disposable. Actuated operation provides a faster material extraction. In the case of a manual operation, the tubular member and the shaft may comprise indicators at the proximal portion to indicate to the user the axial and/or radial position relative to each other. This has the advantage to facilitate to the user the manipulation of the device and specifically provide a more precise axial and/or radial position of the inward and outward projections. Radial position indicators at the proximal portions may be printed or engraved markers or graduated/tapered circular markers. Axial position indicators may be printed or engraved longitudinal indicators and may extend over the surface of the shaft to provide the user information regarding the axial position of the shaft relative to the tubular member.

In the case of actuated operation, the tubular member and/or the shaft may be operated by actuators, e.g., motors, located at the proximal portion of the device. The actuators may be controlled by a computer that is configured to extract material depending on the type of material and the size and characteristics of the extraction site.

The current disclosure may have a broader application that include embodiments for extracting or conveying material such as, soil, concrete, or other muddy substances in a construction environment. Such embodiments may comprise a tubular member with a diameter of about 0.5 m.

The examples and embodiments described herein serve to illustrate rather than limit the invention. The person skilled in the art will be able to design alternative embodiments without departing from the spirit and scope of the present disclosure, as defined by the appended claims and their equivalents. For example, the shape of the inward projections is not restricted to be rectangular or helical. Any type of shape and orientation that allows the shaft to rotate and prevents binding of material on the outward projections remains within the scope of the present disclosure. Reference signs placed in parentheses in the claims shall not be interpreted to limit the scope of the claims. Items described as separate entities in the claims, or the description may be implemented as a single hardware item combining the features of the items described.

1. A device for extracting a material, the device comprising:
a tubular member having a lumen and a series of inward projections extending into the lumen, and
a shaft movably arranged within the lumen of the tubular member, and having a series of outward projections extending into the lumen,
wherein either one of the outward projections and the inward projections are first projections, the first projections having a helical shape with a pitch and a space in axial direction between successive windings of the helical shape, and the other one of the outward projections and the inward projections are second projections,
wherein the second projections are configured to extend at least partially into the space between the successive windings, and wherein first slots between adjacent ones of the first projections allow the second projections to pass through first slots of the first projections and second slots between adjacent ones of the second projections allow the first projections to pass through the second slots of the second projections.

2. The device of claim 1, wherein the second projections are arranged in at least one axially oriented row, wherein the second projections of the row are attached at a same circumferential position on an inner surface of the tubular member or an outer surface of the shaft.

3. The device of claim 1, wherein the first slots between the first projections are arranged in at least one first axially oriented row and the second slots between the second projections are arranged in at least one second axially oriented row.

4. The device of claim 1, wherein the inward and/or outward projections are blades.

5. The device of claim 1, wherein the second projections have a rectangular shape.

6. The device of claim 1, wherein each winding of the helical shape of the first projections is formed by a fixed number of first projections.

7. The device of claim 1, wherein a proximal portion of the tubular member comprises an inner distal annular stopper and a proximal annular stopper to stop, respectively, extension and retraction of an outer ring mounted on the circumferential surface of a proximal portion of the shaft.

8. The device of claim 7, and further comprising an elastic element between one of the annular stoppers of the tubular member and the outer ring of the shaft.

9. The device of claim 1, wherein the second projections have a helical shape with a rotational direction opposite to the rotational direction of the helix of the first projections.

10. The device of claim 9, further comprising an actuated mechanism to rotate the tubular member and shaft with respect to each other around their longitudinal axis.

11. The device of claim 1, wherein the second projections have a projected axial length corresponding substantially to the space in axial direction between the successive first projections.

12. The device of claim 1, wherein the outward projections extend radially outward to substantially cover a radius of the lumen of the tubular member from the outside surface of the shaft to the inner surface of the tubular member or wherein the inward projections extend radially inward to substantially cover the radius of the lumen of the tubular member from the inner surface of the tubular member to the outside surface of the shaft.

13. The device of claim 1, wherein a proximal portion of the tubular member has a side aperture, wherein the inward projections are disposed from a distal end of the tubular member to at least an axial location of the aperture.

14. A method for material transport, the method comprising:
a step of providing a tubular member and a shaft in a lumen of the tubular member, the tubular member having a series of inward projections extending into the lumen, the shaft having a series of outward projections extending into the lumen (22), wherein either one of the outward projections and the inward projections are first projections, the first projections having a helical shape with a pitch and a space in axial direction between successive windings of the helical shape, and the other one of the outward projections and the inward projections are second projections, wherein the second projections extend at least partially into the space between the successive windings, and
a first moving step of moving the shaft with respect to the tubular member so that the first projections pass through second slots between adjacent ones of the second projections.
a second moving step of moving the shaft with respect to the tubular member so that the second projections pass through first slots between adjacent ones of the first projections.

15. The method of claim 14, wherein the first moving step comprises rotating the shaft until the first slots align with the second slots, and then proceeding with the second moving step, or wherein the second moving step comprises moving the shaft until the first slots align with the second slots, and then proceeding with the first step.
